(19) United States
(12) Patent Application Publication
(10) Pub. No.: US 2023/0061740 A1
(43) Pub. Date: Mar. 2, 2023

(54) METHODS AND SYSTEMS FOR MANAGING FLUID PUMP DELIVERY IMPEDIMENTS

(71) Applicant: INSULET CORPORATION, Acton, MA (US)

(72) Inventors: Steven CARDINALI, Tewksbury, MA (US); Ian MCLAUGHLIN, Groton, MA (US)

(21) Appl. No.: 17/822,002

(22) Filed: Aug. 24, 2022

Related U.S. Application Data
(60) Provisional application No. 63/239,092, filed on Aug. 31, 2021.

(51) Int. Cl. A61M 5/145; A61M 5/168

(52) U.S. Cl. CPC A61M 5/1454; A61M 5/16831

(57) ABSTRACT

Fluid delivery devices with occlusion management are described. For example, a fluid delivery system may include at least one force element, at least one moveable element configured to be moved in a dispensing direction by the at least one force element during a pump cycle to expel a fluid from the fluid delivery system, and an occlusion management system comprising at least one emergency element operably coupled to the at least one moveable element, the occlusion management system comprising logic operative to activate the at least one emergency element responsive to a determination of an occlusion to provide additional force to move the at least one moveable element in the dispensing direction. Other embodiments are described.

### RELATED APPLICATIONS

This application claims the benefit of U.S. Provisional Patent Application No. 63/239,092, filed Aug. 31, 2021, the contents of which are incorporated herein by reference in their entirety.

### FIELD OF THE INVENTION

The present disclosure generally relates to a fluid pump system, and, in particular, a fluid pump system configured to detect and manage back pressure, occlusions, or other fluid delivery impediments preventing the free flow of fluid via the fluid pump system.

### BACKGROUND

Healthcare providers may prescribe patients wearable devices for delivering fluids, such as liquid medicaments, as part of a treatment regimen. Non-limiting examples of medicaments may include chemotherapy drugs, hormones (for instance, insulin), pain relief medications, and other types of liquid-based drugs. In general, wearable medicament delivery devices are relatively small form factors that may be adhered to the skin of the patient, with a reservoir to hold the medicament. The device may include a needle or cannula fluidically coupled to the reservoir and extending from the device and into the skin of the patient. A pump may operate to force the fluid from the reservoir, through a fluid path, and out through the needle and into the patient. A control system, with hardware and/or software elements, may be arranged within the device to manage medicament delivery and other device features. The control system may operate alone or in combination with an external computing device, such as a patient smartphone, healthcare provider computer, and/or the like

An operational risk of wearable medicament delivery devices is full or partial pump occlusion that occurs when fluid delivery from the pump is impeded from flowing into the body of the patient, potentially caused by site pressure, scar tissue formation, incorrect cannula insertion, or fluid path blockage. Operation of a wearable medicament delivery device with an occlusion may prevent the patient from receiving a correct dosage and/or may cause the device to become unusable. Conventional wearable medicament delivery devices are either not capable of detecting occlusions or, if an occlusion is detected, do not have a mechanism for overcoming them and returning to normal operation. Therefore, a patient may not be aware that an occlusion has occurred (and that they are not receiving a proper dosage) and/or the patient may be required to replace the device prematurely.

### BRIEF DESCRIPTION OF THE DRAWINGS

FIG. 1 illustrates a first exemplary embodiment of an operating environment in accordance with the present disclosure;

FIG. 2 illustrates a second exemplary embodiment of an operating environment in accordance with the present disclosure;

FIG. 3 illustrates a third exemplary embodiment of an operating environment in accordance with the present disclosure;

FIG. 4 illustrates an embodiment of a wearable fluid delivery device in accordance with the present disclosure;

FIG. 5 illustrates an embodiment of a fluid delivery pump in accordance with the present disclosure;

FIG. 6 illustrates an embodiment of operation of the fluid delivery pump of FIG. 5 in accordance with the present disclosure;

FIGS. 7A and 7B illustrate an embodiment of a fluid delivery pump in accordance with the present disclosure;

FIGS. 8A-8C illustrate embodiments of an emergency element configuration in accordance with the present disclosure;

FIG. 9 illustrates a logic flow in accordance with the present disclosure; and

FIG. 10 illustrates a functional block diagram of a system in accordance with the present disclosure.

The drawings are not necessarily to scale. The drawings are merely representations, not intended to portray specific parameters of the disclosure. The drawings are intended to depict example embodiments of the disclosure, and therefore should not be considered as limiting in scope. In the drawings, like numbering represents like elements.

### DETAILED DESCRIPTION

The described technology generally relates to techniques and systems for managing fluid delivery impediments of a fluid pump of a fluid delivery device. In general, a fluid delivery impediment may be an operating condition of a fluid pump in which the free or expected flow of fluid via the pump from a source to a delivery target is diminished or completely prevented. A fluid delivery impediment may be due to various conditions, such as back pressure, occlusions, blockage, device failure, incorrect pump sequencing, and/or the like. Although the terms “occlusion” and “occlusion management” are used in the present disclosure, these are for illustrative purposes only, as embodiments are configured to manage fluid delivery impediments that occur for any reason.

In some embodiments, an occlusion management system may be configured to detect occlusions in a fluid delivery system and initiate one or more emergency elements to overcome the occlusion (for instance, providing extra delivery power to remove a blockage). Fluid delivery pumps need to be designed to overcome back pressure. There may also be transient back pressure spikes that can come from partial or full system occlusions. If a fluid delivery system is designed to always be able to overcome occlusion back pressure at every pulse, then there could be potential power inefficiencies due to delivering extra power when it is not needed. Accordingly, some embodiments provide an occlusion management system configured to supply the extra power to overcome occlusions only when they are detected.

In various embodiments, the occlusion management system may be used within a wearable fluid delivery device for delivering a fluid to a patient. In some embodiments, the fluid may be or may include a medicament. The wearable fluid delivery device may include a reservoir for holding the fluid, a fluid path in fluid communication with the reservoir, a needle (and/or cannula) in fluid communication with the fluid path to deliver the fluid to the patient wearing the wearable fluid delivery device, and a fluid delivery pump configured to force the fluid from the reservoir, through the fluid delivery path, and into the patient via the needle.

In some embodiments, an occlusion management system may be configured to determine a step, process, sequence, or other operational information of a fluid delivery pump. For example, the occlusion management system may operate to detect fluid delivery impediments, such as occlusions. In some embodiments, occlusions or other fluid flow conditions (for instance, incorrect pump sequencing) may be determined by the occlusion management system the same or similar to techniques described in U.S. Pat. No. 10,272,197, titled “Therapeutic Product Delivery Device with Occlusion Detection via Electric Contacts;” U.S. patent application Ser. No. 16/945,246, titled “Methods to Reduce Risk of Occlusions in Insulin Pump Delivery Systems;” U.S. Pat. No. 10,765,807, titled “Fluid Delivery Device with Sensor;” and U.S. Pat. No. 7,887,505, titled “Flow Condition Sensor Assembly for Patient Infusion Device,” all of which are incorporated by reference as if fully set forth in this Detailed Description.

In some embodiments, an occlusion management system may include an emergency element configured to be activated responsive to detection of an occlusion. In various embodiments, the emergency element may provide additional energy, power, force, and/or the like in an attempt to overcome the occlusion. For example, the emergency element may be coupled to a moveable element of the pump, such as a piston, that operates to provide fluid delivery. The emergency element may provide additional force to move the moveable element to overcome the occlusion. In exemplary embodiments, the emergency element may be activated for a threshold duration (for instance, an “emergency threshold” or “occlusion threshold”) that may be determined based on time, pump cycles, pump sequences, portions of pump cycles or sequences, and/or the like. If the emergency element has been activated longer than the emergency threshold, the emergency element may be deactivated, an occlusion alarm may be activated, and/or the fluid delivery device may deactivate fluid delivery.

Accordingly, embodiments may provide multiple technological advantages over conventional systems. In one non-limiting technological advantage, embodiments may provide techniques and systems to detect and overcome occlusions in fluid delivery systems. In another non-limiting technological advantage, embodiments may provide emergency processes for overcoming occlusions that only require energy when managing an occlusion. In a medicament delivery system, a non-limiting technological advantage may include detecting and removing an occlusion to ensure a proper flow of medicament to a patient or, alternatively, alerting a user if an occlusion cannot be removed.

Other embodiments and technological advantages are contemplated in the present disclosure.

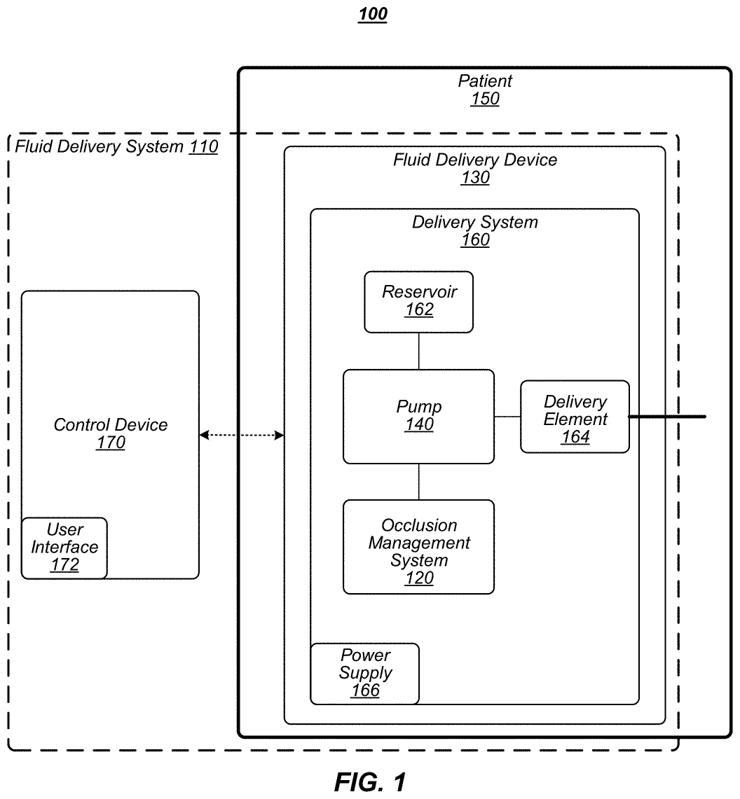

FIG. 1 illustrates an example of an operating environment 100 that may be representative of some embodiments. As shown in FIG. 1, operating environment 100 may include a fluid delivery system 110. In various embodiments, fluid delivery system 110 may include a control or computing device 170 that, in some embodiments, may be communicatively coupled to a fluid delivery device 130. Computing device 170 may be or may include one or more logic devices, including, without limitation, a server computer, a client computing device, a personal computer (PC), a workstation, a laptop, a notebook computer, a smart phone, a tablet computing device, a personal diabetes management (PDM) device, a microcontroller (MCU) and/or the like.

In some embodiments, control device 170 may be in wired or wireless communication with fluid delivery device 130. For example, control device 170 and fluid delivery device 130 may communicate via various wireless protocols, including, without limitation, Wi-Fi (i.e., IEEE 802.11), radio frequency (RF), Bluetooth™, Zigbee™, near field communication (NFC), Medical Implantable Communications Service (MICS), and/or the like. In another example, control device 170 and fluid delivery device 130 may communicate via various wired protocols, including, without limitation, universal serial bus (USB), Lightning, serial, and/or the like. Although control device 170 (and components thereof) and fluid delivery device 130 are depicted as separate devices, embodiments are not so limited. For example, in some embodiments, control device 170 and fluid delivery device 130 may be a single device. In another example, some or all of the components of control device 170 may be included in fluid delivery device 130. For example, fluid delivery device 130 may include processor circuitry, a memory unit, and/or the like. In some embodiments, each of control device 170 and fluid delivery device 130 may include a separate processor circuitry, memory unit, and/or the like capable of facilitating occlusion management processes according to some embodiments, either individually or in operative combination. Embodiments are not limited in this context.

In some embodiments, control device 170 (and/or fluid delivery device 130) may include a user interface 172 configured to present visual information to a user. In various embodiments, the visual information may include operational information associated with fluid delivery device 130. In some embodiments, the visual information may include an occlusion indicator, for instance, to communicate to a user that an occlusion has occurred. In exemplary embodiments, an occlusion indicator may include an alarm that the occlusion could not be resolved by the occlusion management system. In other embodiments, auditory operational information may be provided alone or in addition to the visual information (for instance, an auditory alarm indicating an occlusion condition).

Fluid delivery device 130 may be or may include a wearable automatic fluid delivery device directly coupled to patient 150, for example, directly attached to the skin of the user via an adhesive and/or other attachment component. In some embodiments, fluid delivery device 130 may be or may include a medicament delivery device configured to deliver a liquid medicament, drug, therapeutic agent, or other medical fluid to a patient. Non-limiting examples of medicaments may include insulin, glucagon or a glucagon-like peptide, pain relief drugs, hormones, blood pressure medicines, morphine, methadone, chemotherapy drugs, proteins, antibodies, and/or the like.

In some embodiments, fluid delivery device 130 may be or may include an automatic insulin delivery (AID) device configured to deliver insulin (and/or other medication) to patient 150. For example, fluid delivery device 160 may be or may include a device the same or similar to an OmniPod® device or system provided by Insulet Corporation of Acton, Mass., United States, for example, as described in U.S. Pat. Nos. 7,303,549; 7,137,964; and/or 6,740,059, each of which is incorporated herein by reference in its entirety. Although an AID device and insulin are used in examples in the present disclosure, embodiments are not so limited, as fluid delivery device 130 may be or may include a device capable of storing and delivering any fluid including, without limitation, therapeutic agent, drug, medicine, hormone, protein, antibody, and/or the like.

Fluid delivery device 130 may include one or more housings, a delivery system 160 having a number of components to facilitate automated delivery of a fluid to patient 150, including, without limitation, a reservoir 162 for storing the fluid, a pump 140 for transferring the fluid from reservoir 162, through a fluid path or conduit, and into the body of patient 150, and/or a power supply 166. Fluid delivery device 130 may include at least one delivery element 164, such as a needle and/or cannula, configured to be inserted into the skin of the patient to operate as a conduit between reservoir 162 and patient 150. Embodiments are not limited in this context, for example, as delivery system 162 may include more or fewer components.

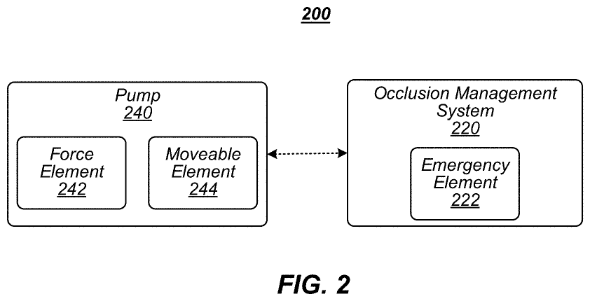

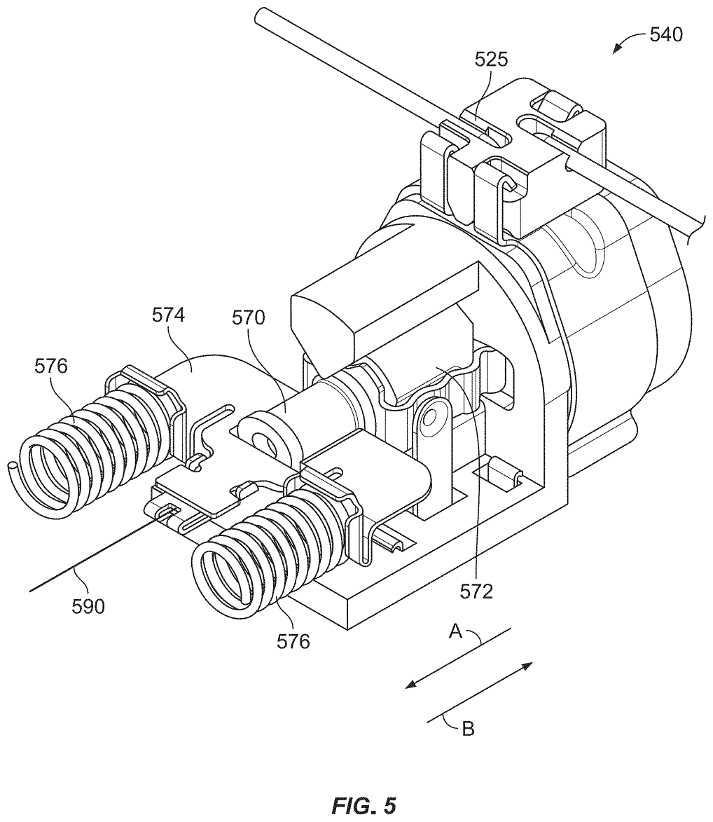

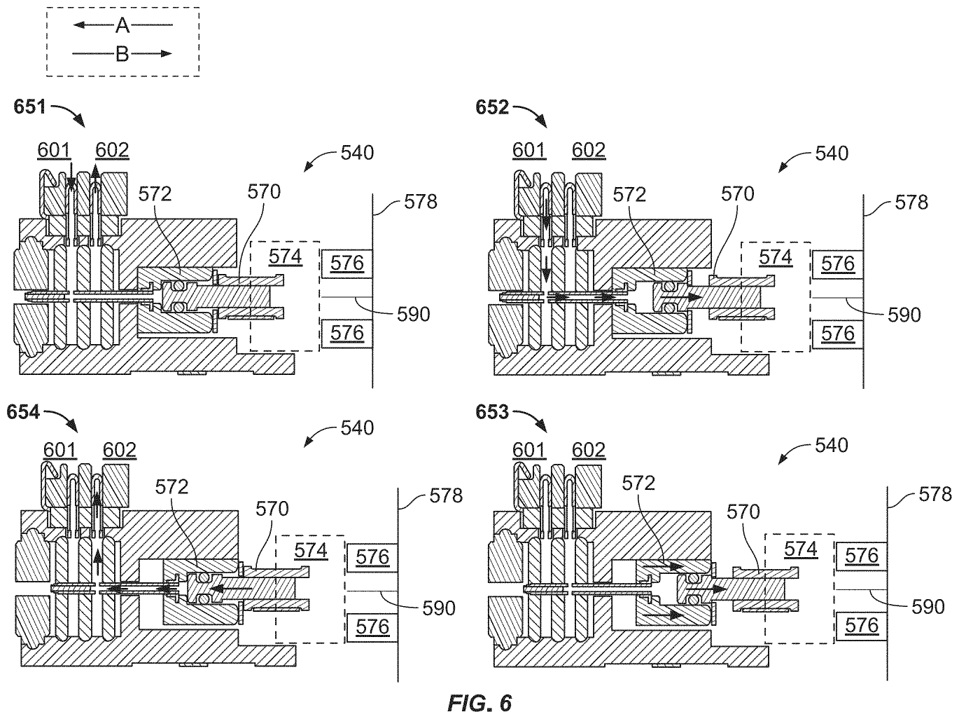

In some embodiments, delivery system 160 may include an occlusion management system 120 operably coupled to pump 140. Occlusion management system 120 may operate to determine a presence of an occlusion through its own control circuitry and/or via control device 170. In some embodiments, occlusion management system 120 may operate to provide additional force to one or more components of pump 140 in an attempt to overcome the occlusion. FIG. 2 illustrates an example of an operating environment 200 that may be representative of some embodiments. As shown in FIG. 2, operating environment 200 may include a pump and an occlusion management system 220. Pump 240 may include a force element 242 configured to force a moveable element 244. In general, a moveable element 244 may include a component of pump 240 that is moved during the fluid pumping process. Non-limiting examples of moveable elements 244 may include a piston, a chamber, a rod, a spring, a wall or other surface, a carrier, and/or the like (see, for example, FIGS. 5 and 6). For example, pump 240 may include a piston that is moved by force element 242 during operation of pump 240. Force element 242 may include a component of pump 240 configured to provide energy, force, and/or the like to moveable element 244 during pump operation. Non-limiting examples of force elements 242 may include a wire, a shape-memory allow (SMA) wire, a rod, a motor, a spring, and/or the like (see, for example, FIGS. 5 and 6).

For example, pump 240 may include an SMA wire force element 242 operative to cause movement of a piston moveable element 244 during a pump sequence of pump 240. When an occlusion occurs, the power provided by force element 242 may not be sufficient to force moveable element 244 as required for the pump sequence. Accordingly, in some embodiments, occlusion management system 220 may include an emergency element 222 configured to provide additional force in an attempt to force moveable element 244 through the pump sequence and, furthermore, to remove the occlusion. In some embodiments, emergency element 222 may be or may include a wire, an SMA wire, a motor, a spring, and/or the like. In various embodiments, emergency element 222 may include an SMA wire.

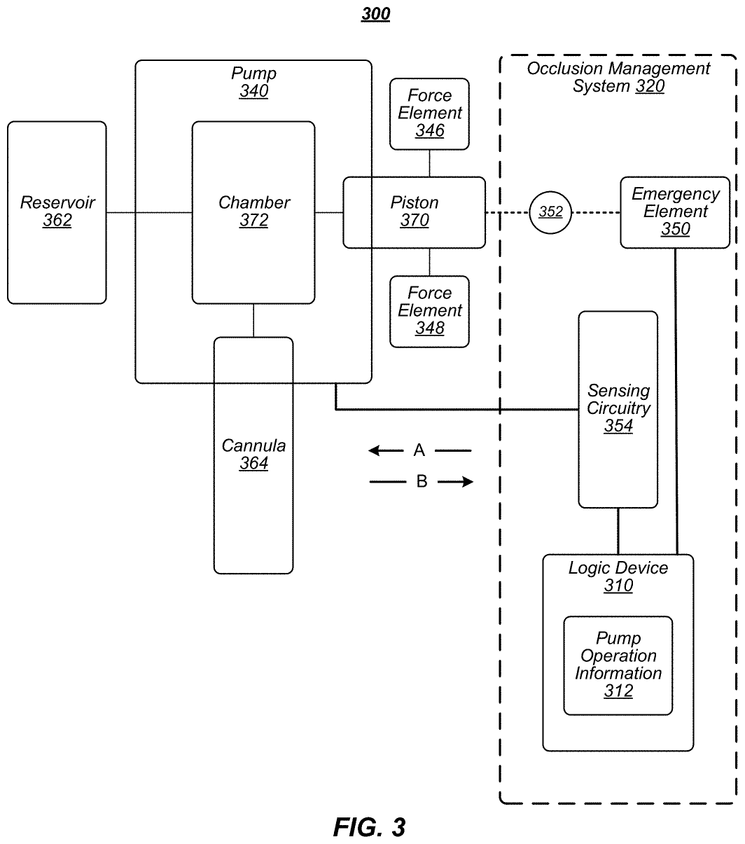

FIG. 3 illustrates an example of an operating environment 300 that may be representative of some embodiments. As shown in FIG. 3, a pump 330 may include a pump 340 having a piston 370 operably coupled to a chamber 372. During a pump cycle, a fluid path (not shown) of chamber 372 may be open to reservoir 362 and closed to cannula 364. A force element 346 may force piston 370 in a first direction (for instance, direction A) to draw fluid from reservoir 362 into chamber 372. In some embodiments, force element 346 may be an SMA wire. Non-limiting examples of SMA wires may include nickel-titanium wires, nitinol wires, alloys thereof, and/or the like. The fluid path of chamber 372 may then be closed to reservoir 362 and opened to cannula 364. Force element 346 may force piston 370 in a second direction (for instance, direction B) to expel the fluid from chamber 372 and into a patient via cannula 364.

In some embodiments, force element 346 may force piston 370 (and chamber 372) in one direction (for instance, direction A) and another force element 348 may force piston 370 in another direction (for instance, direction B). For example, force element 346 may be an SMA wire and force element 348 may be a spring (see, for example, FIG. 5). SMA wire 346 may be energized to pull piston 370 in direction A. Spring (or springs) 348 may be compressed against a surface when piston 370 moves in direction A. When SMA wire 346 is deactivated, the energy of compressed spring 348 may force piston 370 to move in direction B.

Although SMA wires and springs are used in examples in the present disclosure, embodiments are not so limited, as other types of force elements known in the art or developed in the future may be used in accordance with the present disclosure.

In some embodiments, an occlusion management system 320 may include sensing circuitry 354 formed of hardware and/or software and configured to detect operational aspects of pump 340. A logic or control device 310 may be configured to generate pump operation information 312, for example, based on input from sensing circuitry. Logic device 310 may be configured to detect occlusions directly and/or indirectly by determining that a pump cycle is not proceeding correctly. For example, logic device 310 may monitor pump 340 sequencing. If the sequencing does not happen properly, logic device 310 may determine that an occlusion has occurred.

In some embodiments, logic device 310 may activate emergency element 350 responsive to detecting an occlusion. In various embodiments, logic device 310 may wait for an occlusion threshold before activating emergency element 350. The occlusion threshold may be a minimum amount of time, cycles, or sequences of improper pump 340 operation. Emergency element 350 may include an SMA wire, motor, spring, or other force element operably coupled to piston 370 via a connection 352. For example, emergency element 350 may include an SMA wire configured to be coupled to a carrier that pushes/pulls piston in direction A/B. In another example, emergency element 350 may include a motor device capable of engaging piston 370 directly or indirectly to move piston 370 in one of direction A or B. In exemplary embodiments, emergency element 350 may only be coupled to piston 370 (either directly or indirectly) via connection 352 when an occlusion is detected. For example, responsive to detecting an occlusion, logic device 310 may cause emergency element 350 to be coupled to piston 370 via connection 352 and then to become activated 370 to help move piston 370 (and/or other components, such as chamber 372).

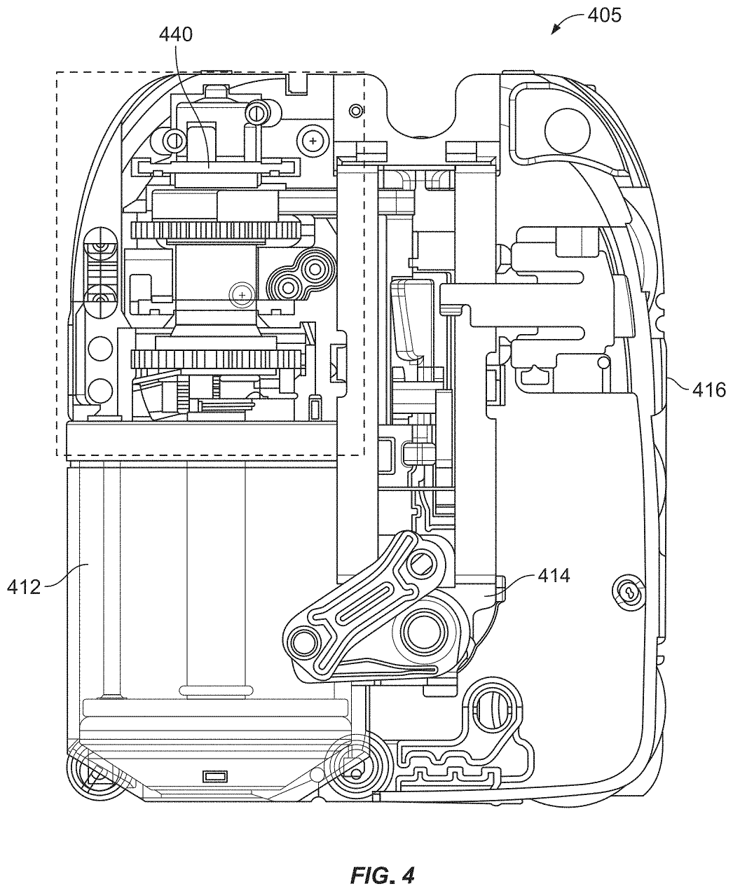

FIG. 4 illustrates an exemplary wearable fluid delivery device in accordance with the present disclosure. In particular, FIG. 4 depicts a top-down view of a wearable fluid delivery device 405. As shown in FIG. 4, a wearable fluid delivery device 405 may include multiple systems to store and deliver a fluid to a patient. In some embodiments, wearable fluid delivery device 405 may include a pump 440. In various embodiments, pump 460 may be or may include a shuttle pump (see, for example, FIG. 5). In exemplary embodiments, wearable fluid delivery device 405 may include a reservoir 412 for storing a fluid. Reservoir may be in fluid communication with pump 440 for delivering the fluid to a patient via needle 414. In some embodiments, components of pump 405 may be arranged within one or more housings 416.

In various embodiments, pump 440 may be a linear volume shuttle pump. In some embodiments, pump 440 may be configured to deliver about 0.5 microliters per pulse. In exemplary embodiments, pump 440 may have a footprint of about 6 millimeters (mm) wide, about 11 mm long, and about 6 mm high. Embodiments are not limited in this context.

FIG. 5 illustrates an embodiment of a fluid delivery pump in accordance with the present disclosure. As shown in FIG. 5, a fluid delivery pump 540 may include springs 576 attached to a carrier 574. Movement of carrier 574 may move piston 570 in one of direction A or B and cause springs 576 to compress or extend.

FIG. 6 illustrates an embodiment of operation of pump 540 in accordance with the present disclosure. As shown in FIG. 6, step 651 shows pump 540 in an initial or “home” position with a fluid path of chamber open to reservoir 601 and closed to patient 602. At step 652, actuator 590 (for instance, an SMA wire or aspiration wire is energized)) may pull piston 470 in direction B to draw fluid into the pump chamber 472. Movement of piston 570 causes carrier 574 to also move in direction B, compressing springs 576 against a wall or other surface 578.

At step 653, actuator 590 moves piston 570 further in direction B, causing piston 570 to engage with an inner wall of chamber 572 and pull chamber in direction B to change the fluid path to be closed to reservoir 601 and open to patient 602. At step 654, actuator 590 is released (for instance, the SMA wire is de-energized) and the compression energy in springs 576 causes them to force piston 570 in direction A to expel the fluid from chamber 572 into the patient via patient fluid path 602. Chamber 572 then moves to the initial position to place pump 540 into the initial state of step 651.

A fluid delivery pump, such as pump 540, may be impeded at various segments of a pump cycle for various reasons. In a first example, a fluid delivery device may be deployed with a flexible cannula inserted in the user. The deployed fluid delivery device, while initially affixed to a site on a user, may shift which may result in movement of the drug delivery device. However, the flexible cannula cannot move since the flexible cannula is inserted in the user. This relative movement between the drug delivery device and the flexible cannula may cause a kink in the flexible cannula. As a result of the kink, an occlusion may occur, for instance, and the full amount of the predetermined amount of insulin may not be delivered within a set period of time.

In a second example, pressure may be applied on a surface of the fluid delivery device (such as the top or the side) and consequently to the site of the body at which the fluid delivery device is located (for instance, a user sleeping on the fluid delivery device). In response to the increased pressure on the fluid delivery device and the site of the body, interstitial fluid may build up within a part of the fluid delivery path of the pump which may cause a back pressure into the cannula (or even further up the fluid delivery path toward the reservoir and pump). As a result, the pump has to work harder to expel both the built-up interstitial fluid and the predetermined amount of fluid.

As a third example, the needle insertion component is operable to puncture the skin of the user with the needle to a depth of the subcutaneous region of the user's skin. The needle is hollow and within the needle is the flexible cannula. The needle insertion component is further operable to retract the needle from the user's skin and leave the flexible cannula within the skin to allow delivery of fluid. However, when the needle is inserted in the skin, the needle may leave punctured tissue below the open end of the cannula from which insulin is output to the user's subcutaneous region. The punctured tissue allows the end of the cannula to freely move up and down and not be occluded. However, external pressure to the skin in the area of the fluid delivery device may inadvertently push the cannula against the non-punctured skin around or below the open end of the cannula. As a result, the cannula may be forced into contact with the non-punctured skin around or below the cannula which may occlude the cannula. Alternatively, or in addition, the punctured skin may heal around the cannula and seal the cannula, thereby causing the occlusion.

In a fourth example, the body's immune system may attack the location of the needle insertion (i.e., fluid infusion site). The human body's immune response may cause inflammation in the area around the cannula within the body and scar tissue may form, which may cause an occlusion or partial occlusion around the cannula and an occlusion of the pump.

Occlusions may also occur when fluid is not absorbed quickly enough by the subcutaneous tissues of the user. The delay in absorption may be related to a number of issues, such as the buildup of scar tissue if the pump is placed at a frequently used location on the user's body. Pump occlusion may be caused by different conditions. Methods and systems operable to mitigate the occurrence of a pump occlusion according to some embodiments provides technological advantages and improvements over presently available fluid delivery systems, including wearable fluid delivery systems.

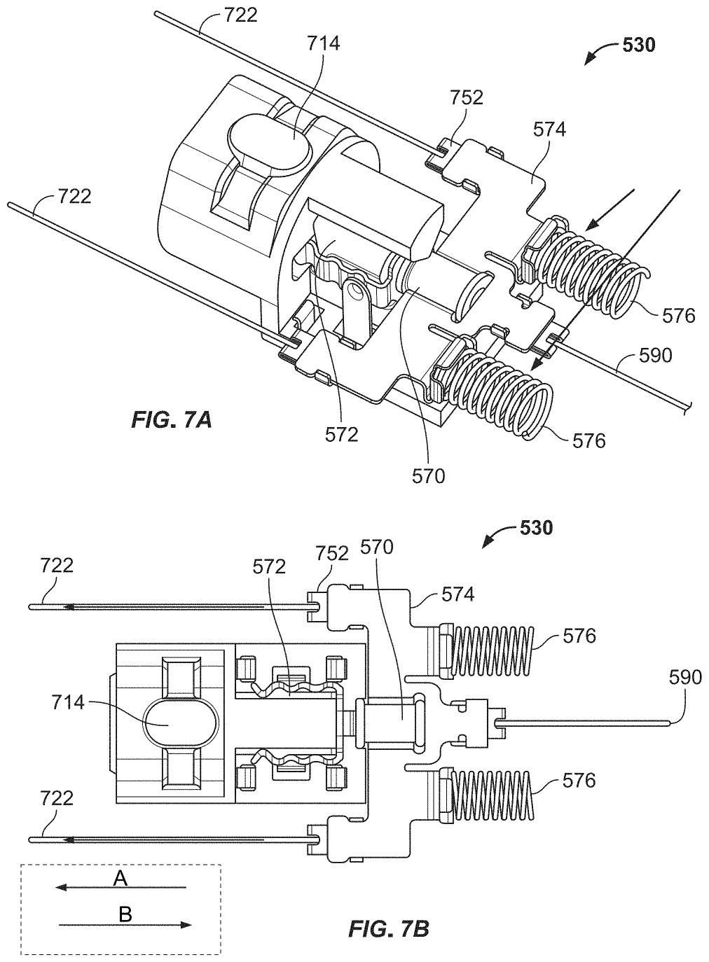

FIGS. 7A and 7B illustrates an embodiment of a fluid delivery pump in accordance with the present disclosure. More specifically, FIG. 7A depicts a bottom perspective view of fluid delivery pump 530 and FIG. 7B depicts a bottom view of fluid delivery pump 530 augmented with an emergency element 722. As shown in FIGS. 7A and 7B, an emergency element in the form of SMA wires 722 may be coupled to carrier 574 via a connector. If an occlusion occurs and piston 570 does not adequately move, for example, in direction A to expel fluid from chamber 572 and into patient via a cannula extending from septa 714, SMA wires 722 may be activated to pull carrier 574, and therefore piston 570, in direction A.

Although two SMA wires 722 are depicted in FIGS. 7A and 7B, embodiments are not so limited, as more or fewer SMA wires 722 may be used. For example, one SMA wire 722 may be used according to some embodiments. In various embodiments, SMA wires 722 may be coupled to other or different components of pump 530, such as piston 570 and/or chamber 572. In exemplary embodiments, SMA wires 722 may be of sufficient length so that they are not pulled or stretched during normal pump operation. Rather, SMA wires 722 may be sized, positioned, and/or configured (for instance, coiled) so that they are only pulled or stretched when activated.

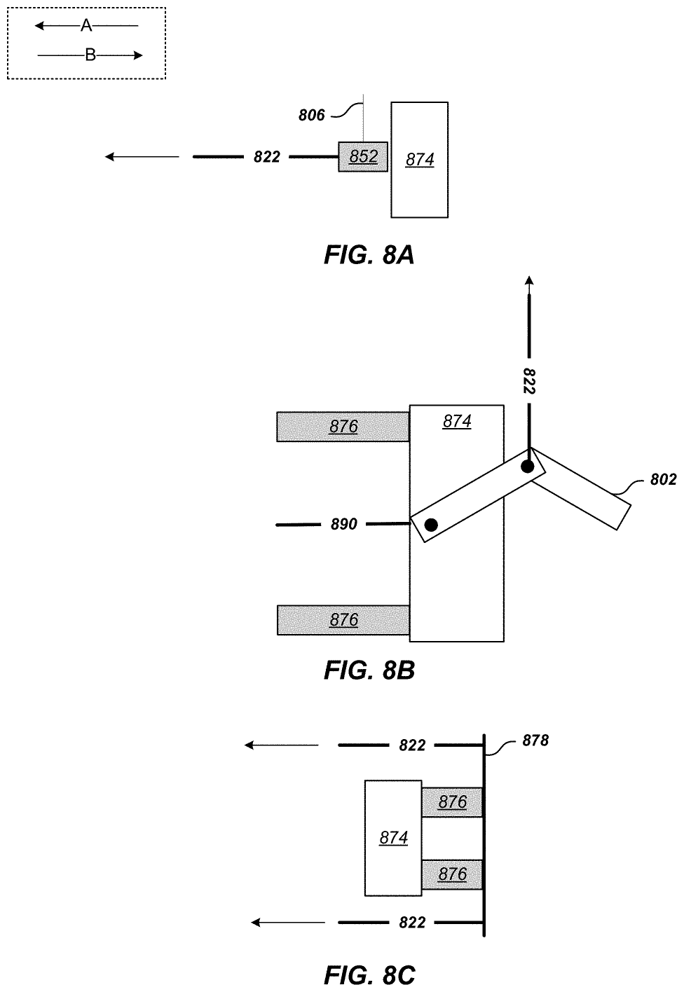

FIGS. 8A-8C illustrate embodiments of an emergency element configuration in accordance with the present disclosure. In some embodiments, an emergency element may be coupled to a component of a pump, such as a carrier, piston, and/or the like to provide additional pushing/pulling force. In various embodiments, the emergency element may be disengaged from the component until needed and activated, which prevents the emergency element from being pulled/stretched with every pump pulse when not needed. Referring to FIG. 8A, emergency element (SMA wire) 822 may be configured to be coupled to carrier 874 via connector 852. In some embodiments, connector 852 may be a latch, snap, or other structure that connects emergency element 822 to carrier 874. For example, a connector SMA wire 806 may be activated in response to a determination that there is an occlusion, which causes connector 852 to become coupled to carrier 874.

Referring to FIG. 8B, in some embodiments, an actuating or aspiration SMA wire 890 may be configured to pull carrier 874 in direction A, to be returned in direction B via springs 876. A linkage 802, such as a two-bar linkage may be operably coupled to carrier 874 at a first end and a wall portion of a pump, or other surface at an opposite second end. Emergency SMA wire 822 may be coupled to a knee or pivot point of linkage 802 to pull on linkage 802 to cause a corresponding movement of carrier 874 (for instance, in direction B). In various embodiments, the first end of two-bar linkage 802 may be arranged on a portion of carrier 874 formed between springs 876. In some embodiments, emergency SMA wire 822 may extend perpendicular to aspiration SMA wire 890 and could fire upon detection of an occlusion, for example, when two-bar linkage 802 is nearly linear, yielding the greatest mechanical advantage.

Referring to FIG. 8C, in some embodiments, emergency SMA wires 822 may be configured to pull a movable wall 878 or other surface in direction A (i.e., to move piston to cause expulsion of fluid). For example, emergency SMA wires 822 may be attached to wall 878 that springs 876 are attached to. Wall 878 would be movable in a direction toward the pump body. Upon detection of an occlusion, actuating emergency SMA wires 822 may pull wall 878 toward pump and cause springs 876 to compress and force the piston forward (i.e., direction A), in an attempt to dislodge the occlusion.

Included herein are one or more logic flows representative of exemplary methodologies for performing novel aspects of the disclosed architecture. While, for purposes of simplicity of explanation, the one or more methodologies shown herein are shown and described as a series of acts, those skilled in the art will understand and appreciate that the methodologies are not limited by the order of acts. Some acts may, in accordance therewith, occur in a different order and/or concurrently with other acts from that shown and described herein. For example, those skilled in the art will understand and appreciate that a methodology could alternatively be represented as a series of interrelated states or events, such as in a state diagram. Moreover, not all acts illustrated in a methodology may be required for a novel implementation.

A logic flow may be implemented in software, firmware, hardware, or any combination thereof. In software and firmware embodiments, a logic flow may be implemented by computer executable instructions stored on a non-transitory computer readable medium or machine readable medium. Embodiments are not limited in this context.

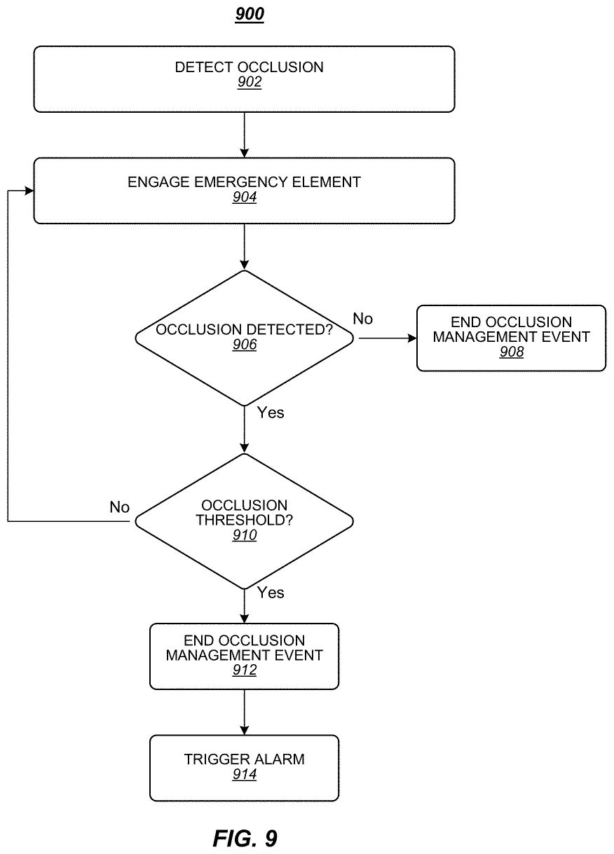

FIG. 9 illustrates an embodiment of a logic flow 900. Logic flow 900 may be representative of some or all of the operations executed by one or more embodiments described herein, such as devices of operating environment 100 and 300. For example, logic flow 400 may be a process performed by controller 170 of FIG. 1 and/or logic device 310 of FIG. 3.

At block 902, logic flow 900 may detect an occlusion. For example, logic device 310 may determine that a full or partial occlusion has occurred due to abnormal operation of pump 340 based on pump operation information 312. In another example, an occlusion may be detected using a pressure sensor and/or a force sensor. For instance, an increased pressure and/or force compared with normal operating conditions may be detected in the pump 340, driving mechanism, SMA wire, and/or the like. An increased pressure and/or force over a threshold may indicate an occlusion. In general, there may be different types of detected occlusions, for example, a full occlusion where the pump cannot finish a pump cycle or a partial occlusion where the pump may finish the pump cycle, but it takes more time, force, pressure, and/or the like to complete.

Logic flow 900 may engage a collusion management element at block 904. For example, logic device 310 may activate emergency element 350 to assist piston 370 (and, in some embodiments, chamber 372) with moving during a pump cycle.

In some embodiments, an emergency element 350 may not take over an entire stroke or cycle; rather, emergency element 350 may just finish the stroke (with high mechanical advantage). For instance, a primary actuation element may go a partial stroke (due to an occlusion), then emergency element 350 may be engaged if primary wire doesn't finish in expected time (or a number of failings). In some embodiments, the timing of an engagement or activation of an emergency element 350 may be associated with the clock of the pump cycle, for example, so that the emergency element 350 is pulling/pushing at the appropriate time in the pump cycle. In various embodiments, for example, an emergency element (SMA wire) activation may be about 0.25 seconds.

At block 906, logic flow 900 may determine whether an occlusion is detected in a subsequent cycle. If no occlusion is detected, logic flow 900 may end the occlusion management event at block 908. For example, the emergency element may be disengaged and/or deactivated. If an occlusion is detected at block 906, logic flow 900 may determine whether the occlusion threshold has been met at block 910. For example, the emergency element may be activated for a threshold duration that may be determined based on time, pump cycles, pump sequences, portions of pump cycles or sequences, and/or the like. If the occlusion threshold has not been met, logic flow 900 may maintain engagement and/or activation of the emergency element. If the occlusion threshold has been met, logic flow may end the occlusion management event at block 912. For example, the emergency element may be disengaged and/or deactivated. At block 914, an alarm may be triggered by logic flow 900. For example, if the emergency element has been activated longer than the emergency threshold, the emergency element may be deactivated, an occlusion alarm may be activated, and/or the fluid delivery device may deactivate fluid delivery.

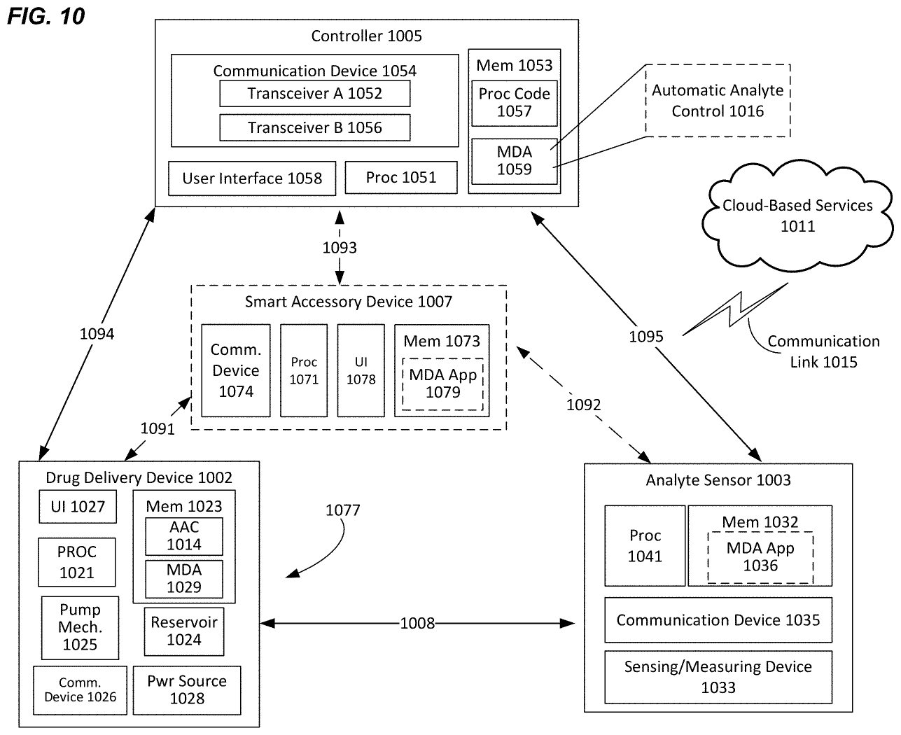

FIG. 10 illustrates a functional block diagram of a system example suitable for implementing the example processes and techniques described herein.

The operating environment 1000 may be or may include an automatic drug delivery system that may include components such as an automatic drug delivery system that is configured to determine a drug dosage and deliver the dosage of the drug without any user interaction, or in some examples, limited user interaction, such as in response to a user depressing a button to indicate measurement of blood glucose or another analyte, or the like. “Drug delivery system environment” may refer to a computing and sensing environment that includes cloud based services, a drug delivery system (that may include a controller, a drug delivery device, and an analyte sensor) and optionally additional devices. The components of the drug delivery system environment may cooperate to provide present analyte measurement values or at least accurate estimates of present analyte measurement values to facilitate calculation of optimal drug dosages for a user.

The automatic drug delivery system 1000 may implement (and/or provide functionality for) a medication delivery algorithm, such as an artificial pancreas (AP) application, to govern or control automated delivery of a drug or medication, such as insulin, to a user (e.g., to maintain euglycemia—a normal level of glucose in the blood). The drug delivery system 1000 may be an automated drug delivery system that may include a wearable automatic drug delivery device 1002, an analyte sensor 1003, and a management device (for instance, a PDM, smart phone, table computing device, and/or the like) 1005.

The system 1000, in an optional example, may also include a smart accessory device 1007, such as a smartwatch, a personal assistant device or the like, which may communicate with the other components of system 1000 via either a wired or wireless communication links 1091-1093.

The management device 1005 may be a computing device such as a smart phone, a tablet, a personal diabetes management device, a dedicated diabetes therapy management device, or the like. In an example, the management device (PDM) 1005 may include a processor 1051, a management device memory 1053, a user interface 1058, and a communication device 1054. The management device 1005 may contain analog and/or digital circuitry that may be implemented as a processor 1051 for executing processes based on programming code stored in the management device memory 1053, such as the medication delivery algorithm or application (MDA) 1059, to manage a user's blood glucose levels and for controlling the delivery of the drug, medication, or therapeutic agent to the user as well as other functions, such as calculating carbohydrate-compensation dosage, a correction bolus dosage and the like as discussed above. The management device 1005 may be used to program, adjust settings, and/or control operation of the wearable automatic drug delivery device 1002 and/or the analyte sensor 1003 as well as the optional smart accessory device 1007.

The processor 1051 may also be configured to execute programming code stored in the management device memory 1053, such as the MDA 1059. The MDA 1059 may be a computer application that is operable to deliver a drug based on information received from the analyte sensor 1003, the cloud-based services 1011 and/or the management device 1005 or optional smart accessory device 1007. The memory 1053 may also store programming code to, for example, operate the user interface 1058 (e.g., a touchscreen device, a camera or the like), the communication device 1054 and the like. The processor 1051 when executing the MDA 1059 may be configured to implement indications and notifications related to meal ingestion, blood glucose measurements, and the like. The user interface 1058 may be under the control of the processor 1051 and be configured to present a graphical user interface that enables the input of a meal announcement, adjust setting selections and the like as described above.

In a specific example, when the MDA 1059 is an artificial pancreas (AP) application, the processor 1051 is also configured to execute a diabetes treatment plan (which may be stored in a memory) that is managed by the MDA 1059 stored in memory 1053. In addition to the functions mentioned above, when the MDA 1059 is an AP application, it may further provide functionality to enable the processor 1051 to determine a carbohydrate-compensation dosage, a correction bolus dosage and determine a basal dosage according to a diabetes treatment plan. In addition, as an AP application, the MDA 1059 provides functionality to enable the processor 1051 to output signals to the wearable automatic drug delivery device 1002 to deliver determined bolus and basal dosages.

The communication device 1054 may include one or more transceivers such as Transceiver A 1052 and Transceiver B 1056 and receivers or transmitters that operate according to one or more radio-frequency protocols. In the example, the transceivers 1052 and 1056 may be a cellular transceiver and a Bluetooth® transceiver, respectively. For example, the communication device 1054 may include a transceiver 1052 or 1056 configured to receive and transmit signals containing information usable by the MDA 1059.

The wearable automatic drug delivery device 1002, in the example system 1000, may include a user interface 1027, a controller 1021, a drive mechanism 1025, a communication device 1026, a memory 1023, a power source/energy harvesting circuit 1028, device sensors 1084, and a reservoir 1024. The wearable automatic drug delivery device 1002 may be configured to perform and execute the processes described in the examples of the present disclosure without input from the management device 1005 or the optional smart accessory device 1007. As explained in more detail, the controller 1021 may be operable, for example, to determine an amount of insulin delivered, IOB, insulin remaining, and the like. The controller 1021 alone may determine an amount of insulin delivered, IOB, insulin remaining, and the like, such as control insulin delivery, based on an input from the analyte sensor 1004.

The memory 1023 may store programming code executable by the controller 1021. The programming code, for example, may enable the controller 1021 to control expelling insulin from the reservoir 1024 and control the administering of doses of medication based on signals from the MDA 1029 or, external devices, if the MDA 1029 is configured to implement the external control signals.

The reservoir 1024 may be configured to store drugs, medications or therapeutic agents suitable for automated delivery, such as insulin, morphine, blood pressure medicines, chemotherapy drugs, or the like.

The device sensors 1084 may include one or more of a pressure sensor, a power sensor, or the like that are communicatively coupled to the controller 1021 and provide various signals. For example, the pressure sensor may be coupled to or integral with a needle/cannula insertion component (which may be part of the drive mechanism 1025) or the like. In an example, the controller 1021 or a processor, such as 1051, may be operable to determine that a rate of drug infusion based on the indication of the fluid pressure. The rate of drug infusion may be compared to an infusion rate threshold, and the comparison result may be usable in determining an amount of insulin onboard (OOB) or a total daily insulin (TDI) amount.

In an example, the wearable automatic drug delivery device 1002 includes a communication device 1026, which may be a receiver, a transmitter, or a transceiver that operates according to one or more radio-frequency protocols, such as Bluetooth, Wi-Fi, a near-field communication standard, a cellular standard, or the like. The controller 1021 may, for example, communicate with a personal diabetes management device 1005 and an analyte sensor 1003 via the communication device 1026.

The wearable automatic drug delivery device 1002 may be attached to the body of a user, such as a patient or diabetic, at an attachment location and may deliver any therapeutic agent, including any drug or medicine, such as insulin or the like, to a user at or around the attachment location. A surface of the wearable automatic drug delivery device 1002 may include an adhesive to facilitate attachment to the skin of a user as described in earlier examples.

The wearable automatic drug delivery device 1002 may, for example, include a reservoir 1024 for storing the drug (such as insulin), a needle or cannula (not shown in this example) for delivering the drug into the body of the user (which may be done subcutaneously, intraperitoneally, or intravenously), and a drive mechanism 1025 for transferring the drug from the reservoir 1024 through a needle or cannula and into the user. The drive mechanism 1025 may be fluidly coupled to reservoir 1024, and communicatively coupled to the controller 1021.

The wearable automatic drug delivery device 1002 may further include a power source 1028, such as a battery, a piezoelectric device, other forms of energy harvesting devices, or the like, for supplying electrical power to the drive mechanism 1025 and/or other components (such as the controller 1021, memory 1023, and the communication device 1026) of the wearable automatic drug delivery device 1002.

In some examples, the wearable automatic drug delivery device 1002 and/or the management device 1005 may include a user interface 1058, respectively, such as a keypad, a touchscreen display, levers, light-emitting diodes, buttons on a housing of the management device 1005, a microphone, a camera, a speaker, a display, or the like, that is configured to allow a user to enter information and allow the management device 1005 to output information for presentation to the user (e.g., alarm signals or the like). The user interface 1058 may provide inputs, such as a voice input, a gesture (e.g., hand or facial) input to a camera, swipes to a touchscreen, or the like, to processor 1051 which the programming code interprets.

When configured to communicate to an external device, such as the PDM 1005 or the analyte sensor 1004, the wearable automatic drug delivery device 1002 may receive signals over the wired or wireless link 1094 from the management device (PDM) 1005 or 1008 from the analyte sensor 1004. The controller 1021 of the wearable automatic drug delivery device 1002 may receive and process the signals from the respective external devices as described with reference to the examples of the present disclosure as well as implementing delivery of a drug to the user according to a diabetes treatment plan or other drug delivery regimen.

The smart accessory device 1007 may be, for example, an Apple Watch®, other wearable smart device, including eyeglasses, provided by other manufacturers, a global positioning system-enabled wearable, a wearable fitness device, smart clothing, or the like. Similar to the management device 1005, the smart accessory device 1007 may also be configured to perform various functions including controlling the wearable automatic drug delivery device 1002. For example, the smart accessory device 1007 may include a communication device 1074, a processor 1071, a user interface 1078 and a memory 1073. The user interface 1078 may be a graphical user interface presented on a touchscreen display of the smart accessory device 1007. The memory 1073 may store programming code to operate different functions of the smart accessory device 1007 as well as an instance of the MDA 1079. The processor 1071 that may execute programming code, such as site MDA 1079 for controlling the wearable automatic drug delivery device 1002 to implement the FIGS. 3, 7A, and 7B examples described herein.

The analyte sensor 1003 may include a controller 1031, a memory 1032, a sensing/measuring device 1033, a user interface 1037, a power source/energy harvesting circuitry 1034, and a communication device 1035. The analyte sensor 1003 may be communicatively coupled to the processor 1051 of the management device 1005 or controller 1021 of the wearable automatic drug delivery device 1002. The memory 1032 may be configured to store information and programming code, such as an instance of the MDA 1036.

The analyte sensor 1003 may be configured to detect multiple different analytes, such as lactate, ketones, uric acid, sodium, potassium, alcohol levels or the like, and output results of the detections, such as measurement values or the like. The analyte sensor 1003 may, in an example, be configured to measure a blood glucose value at a predetermined time interval, such as every 5 minutes, every cycle, or the like. The communication device 1035 of analyte sensor 1003 may have circuitry that operates as a transceiver for communicating the measured blood glucose values to the management device 1005 over a wireless link 1095 or with wearable automatic drug delivery device 1002 over the wireless communication link 1008. While called an analyte sensor 1003, the sensing/measuring device 1033 of the analyte sensor 1003 may include one or more additional sensing elements, such as a glucose measurement element a heart rate monitor, a pressure sensor, or the like. The controller 1031 may include discrete, specialized logic and/or components, an application-specific integrated circuit, a microcontroller or processor that executes software instructions, firmware, programming instructions stored in memory (such as memory 1032), or any combination thereof.

Similar to the controller 1021, the controller 1031 of the analyte sensor 1003 may be operable to perform many functions. For example, the controller 1031 may be configured by the programming code stored in the memory 1032 to manage the collection and analysis of data detected the sensing and measuring device 1033.

Although the analyte sensor 1003 is depicted in FIG. 10 as separate from the wearable automatic drug delivery device 1002, in various examples, the analyte sensor 1003 and wearable automatic drug delivery device 1002 may be incorporated into the same unit. That is, in various examples, the sensor 1003 may be a part of the wearable automatic drug delivery device 1002 and contained within the same housing of the wearable automatic drug delivery device 1002 (e.g., the sensor 1003 or, only the sensing/measuring device 1033 and memory storing related programming code may be positioned within or integrated into, or into one or more components, such as the memory 1023, of, the wearable automatic drug delivery device 1002). In such an example configuration, the controller 1021 may be able to implement the process examples of present disclosure alone without any external inputs from the management device 1005, the cloud-based services 1011, another sensor (not shown), the optional smart accessory device 1007, or the like.

The communication link 1015 that couples the cloud-based services 1011 to the respective devices 1002, 1003, 1005 or 1007 of system 1000 may be a cellular link, a Wi-Fi link, a Bluetooth link, or a combination thereof. Services provided by cloud-based services 1011 may include data storage that stores anonymized data, such as blood glucose measurement values, historical IOB or TDI, prior carbohydrate-compensation dosage, and other forms of data. In addition, the cloud-based services 1011 may process the anonymized data from multiple users to provide generalized information related to TDI, insulin sensitivity, IOB and the like.

The wireless communication links 1008, 1091, 1092, 1093, 1094 and 1095 may be any type of wireless link operating using known wireless communication standards or proprietary standards. As an example, the wireless communication links 1008, 1091, 1092, 1093, 1094 and 1095 may provide communication links based on Bluetooth®, Zigbee®, Wi-Fi, a near-field communication standard, a cellular standard, or any other wireless protocol via the respective communication devices 1054, 1074, 1026 and 1035.

Software related implementations of the techniques described herein may include, but are not limited to, firmware, application specific software, or any other type of computer readable instructions that may be executed by one or more processors. The computer readable instructions may be provided via non-transitory computer-readable media. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AP application and algorithms may be operable to perform various functions related to open loop operations, such as the generation of prompts requesting the input of information such as weight or age. Similarly, a dosage amount of insulin may be received by the AP application or algorithm from a user via a user interface. Other open-loop actions may also be implemented by adjusting user settings or the like in an AP application or algorithm.

Some examples of the disclosed device or processes may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

1. A fluid delivery system, comprising:
at least one force element;
at least one moveable element configured to be moved in a dispensing direction by the at least one force element during a pump cycle to expel a fluid from the fluid delivery system; and
an occlusion management system comprising at least one emergency element operably coupled to the at least one moveable element, the occlusion management system comprising logic operative to activate the at least one emergency element responsive to a determination of an occlusion to provide additional force to move the at least one moveable element in the dispensing direction.

2. The fluid delivery system of claim 1, the at least one force element comprising at least one spring.

3. The fluid delivery system of claim 1, the at least one moveable element comprising a piston.

4. The fluid delivery system of claim 1, the at least one emergency element comprising at least one of a shape memory allow (SMA) wire, a motor, or a spring.

5. The fluid delivery system of claim 1, the at least one emergency element configured to be operably disconnected from the at least one moveable element when deactivated, and operably coupled to the at least one moveable element when activated.

6. The fluid delivery system of claim 1, further comprising at least one two-bar link configured to operably couple the at least one emergency element and the at least one moveable element.

7. The fluid delivery system of claim 1, the at least one emergency element operably coupled to a moveable wall, wherein activation of the at least one emergency element operates to move the movable wall in the dispensing direction.

8. The fluid delivery system of claim 1, the logic to determine an occlusion threshold for activation of the at least one emergency element.

9. The fluid delivery system of claim 8, the occlusion threshold based on one of a number of pump cycles or a timed duration.

10. The fluid delivery system of claim 8, the logic to, responsive to expiry of the occlusion threshold, perform one or more of initiating an alarm or stopping performance of pump cycles.

11. A method, comprising:
monitoring for an occlusion of a fluid delivery device comprising:
at least one force element,
at least one moveable element configured to be moved in a dispensing direction by the at least one force element during a pump cycle to expel a fluid from the fluid delivery system, and
an occlusion management system comprising at least one emergency element operably coupled to the at least one moveable element, the occlusion management system; and
activate the at least one emergency element responsive to a determination of the occlusion to provide additional force to move the at least one moveable element in the dispensing direction.

12. The method of claim 11, the at least one force element comprising at least one spring.

13. The method of claim 11, the at least one moveable element comprising a piston.

14. The method of claim 11, the at least one emergency element comprising at least one of a shape memory allow (SMA) wire, a motor, or a spring.

15. The method of claim 11, the at least one emergency element configured to be operably disconnected from the at least one moveable element when deactivated, and operably coupled to the at least one moveable element when activated.

16. The method of claim 11, fluid delivery device further comprising at least one two-bar link configured to operably couple the at least one emergency element and the at least one moveable element.

17. The method of claim 11, the at least one emergency element operably coupled to a moveable wall, wherein activation of the at least one emergency element operates to move the movable wall in the dispensing direction.

18. The method of claim 11, comprising determining an occlusion threshold for activation of the at least one emergency element.

19. The method of claim 18, the occlusion threshold based on one of a number of pump cycles or a timed duration.

20. The method of claim 18, comprising, responsive to expiry of the occlusion threshold, performing one or more of initiating an alarm or stopping performance of pump cycles.
